(19) United States
(12) Patent Application Publication
(10) Pub. No.: US 2003/0119178 A1
(43) Pub. Date: Jun. 26, 2003

(54) DNA chip and its preparation

(76) Inventors: Kozo Sato, Kanagawa (JP); Koki Nakamura, Kanagawa (JP); Hiroshi Shinoki, Saitama (JP)

Correspondence Address: REED SMITH, LLP, ATTN: PATENT RECORDS DEPARTMENT, 599 LEXINGTON AVENUE, 29TH FLOOR, NEW YORK, NY, 10022-7650, US

(21) Appl. No.: 10/308,689

(22) Filed: Dec. 3, 2002

Related U.S. Application Data
(62) Division of application No. 09/749,137, filed on Dec. 27, 2000.

(30) Foreign Application Priority Data
Dec. 27, 1999 (JP) 11-370182

(51) Int. Cl. C12M 1/34; B05D 3/00

(52) U.S. Cl. 435/287.2; 427/2.11

(57) ABSTRACT

An analytical element (typically DNA chip) composed of a solid carrier and a group of nucleotide derivatives or their analogues fixed to the solid carrier via covalent bonding containing a boron-containing ring structure can be favorably prepared by bringing in a liquid phase a group of nucleotide derivatives or their analogues having at one end or its vicinity a boron-containing group or a divalent group reactive to the boron-containing group to produce a boron-containing ring structure into contact with a solid carrier having on its surface a divalent group reactive to the boron-containing group or a boron-containing group, respectively.

### FIELD OF THE INVENTION

This invention relates to a solid carrier to which nucleotide derivatives or their analogues (e.g., oligonucleotides, polynucleotides, and peptide-nucleotides) are attached, which is generally named DNA chip and which is favorably employable for detecting, with high sensitivity, complementary nucleic acid fragments.

### BACKGROUND OF THE INVENTION

Detection of a nucleic acid fragment is generally performed using a probe oligonucleotide which is complementary to the nucleic acid fragment to be detected, by way of hybridization. The probe oligonucleotide is generally fixed onto a solid carrier (e.g., solid substrate) to produce a so-called DNA chip. In the detection procedures, a nucleic acid fragment in a sample liquid is provided with a fluorescent label or a radioisotope label, and then the sample liquid is brought into contact with the probe oligonucleotide of the DNA chip. If the labelled nucleic acid fragment in the sample liquid is complementary to the probe oligonucleotide, the labelled nucleic acid fragment is combined with the probe oligonucleotide by hybridization. The labelled nucleic acid fragment fixed to the DNA chip by hybridization with the probe oligonucleotide is then detected by an appropriate detection method such as fluorometry or autoradiography. The DNA chip is widely employed in the gene technology, for instance, for detecting a complementary nucleic acid fragment and sequencing the detected nucleic acid fragment.

The DNA chip can be utilized to efficiently detect a large number of complementary nucleic acid fragments in a small amount of a sample liquid within a short period of time.

Detection of nucleic acid fragment using an electrochemical label is also known (Japanese Patent Provisional Publication No. 9-288080, and a preprint of the 57th Analytical Chemistry Conference pp. 137-138 (1996)).

P. E. Nielsen et al., Science, 254, 1497-1500(1991) and P. E. Nielsen et al., Biochemistry, 36, pp.5072-5077 (1997) describe PNA (Peptide Nucleic Acid or Polyamide Nucleic Acid) which has no negative charge and functions in the same manner as DNA fragment does. PNA has a polyamide skeleton of N-(2-aminoethyl)glycine units and has neither glucose units nor phosphate groups.

Since PNA is electrically neutral and is not charged in the absence of an electrolytic salt, PNA is able to hybridize with a complementary nucleic acid fragment to form a hybrid which is more stable than the hybrid structure given by a probe oligonucleotide and its complementary nucleic acid fragment (Preprint of the 74th Spring Conference of Japan Chemical Society, pp. 1287, reported by Naomi Sugimoto).

Japanese Patent Provisional Publication No. 11-332595 describes a PNA probe fixed onto a solid carrier at its one end and a detection method utilizing the PNA probe. The PNA probe is fixed onto the solid carrier by the known combination of avidin and biotin.

The aforementioned P. E. Nielsen et al., Science, 254, 1497-1500(1991) also describes a PNA probe labelled with an isotope element and a detection method of a complementary nucleic acid fragment.

Since the PNA probe shows no electric repulsion to a target nucleic acid fragment in a sample liquid, an improved high detection sensitivity is expected.

At present, two methods are known for preparing a DNA chip having a solid carrier and oligonucleotides or polynucleotides fixed onto the carrier. One preparation method comprises preparing oligonucleotides or polynucleotides, step by step on the carrier. This method is named “on-chip method”. A typical on-chip method is described in Foder, S. P. A., Science, 251, page 767 (1991).

Another preparation method comprises fixing separately prepared oligonucleotides or polynucleotides onto a solid carrier. Various methods are known for various oligonucleotides and polynucleotides.

In the case of the complementary nucleotide derivatives (which are synthesized using mRNA as mold) or PCR products (which are DNA fragments prepared by multiplying cDNA by PCR method), an aqueous solution of the prepared DNA fragment is spotted onto a solid carrier having a poly-cationic coat in a DNA chip-preparing device to fix the DNA fragment to the carrier via electrostatic bonding, and then blocking a free surface of the polycationic coat.

In the case that the oligonucleotides are synthetically prepared and have a functional group, an aqueous solution of the synthetic oligonucleotides is spotted onto an activated or reactive solid carrier to produce covalent bonding between the oligonucleotides and the carrier surface. See Lamture, J. B., et al., Nucl. Acids Res., 22, 2121-2125, 1994, and Guo, Z., et al., Nucl. Acids Res., 22, 5456-5465, 1994. Generally, the oligonucleotides are covalently bonded to the surface activated carrier via linking groups.

Also known is a process comprising the steps of aligning small polyacrylamide gels on a glass plate and fixing synthetic oligonucleotides onto the glass plate by making a covalent bond between the polyacrylamide and the oligonucleotide (Yershov, G., et al., Proc. Natl. Acad. Sci. USA, 94, 4913(1996)). Sosnowski, R. G., et al., Proc. Natl. Acad. Sci. USA, 94, 1119-1123 (1997) discloses a process comprising the steps of placing an array of microelectrodes on a silica chip, forming on the microelectrode a streptoavidin-comprising agarose layer, and attaching biotin-modified DNA fragments to the agarose layer by positively charging the agarose layer. Schena, M., et al., Proc. Natl. Acad. Sci. USA, 93, 10614-10619 (1996) teaches a process comprising the steps of preparing a suspension of an amino group-modified PCR product in SSC (i.e., standard sodium chloride-citric acid buffer solution), spotting the suspension onto a slide glass, incubating the spotted glass slide, treating the incubated slide glass with sodium borohydride, and heating thus treated slide glass.

As is explained above, most of the known methods of fixing separately prepared DNA fragments onto a solid carrier utilize the electrostatic bonding or the covalent bonding such as described above.

In any DNA chips having separately prepared oligonucleotide probes on its solid carrier, the oligonucleotide probes should be firmly fixed onto the carrier, so that the hybridization can proceed smoothly between the fixed oligonucleotide probes and target DNA fragments complementary to the fixed oligonucleotide probes.

Further, it is preferred that a surface area of the solid carrier other than the portion to which the probe oligonucleotides are fixed is inactive to the labelled DNA fragments, so that non-complementary DNA fragments in the liquid sample can be kept from attaching onto the surface in the course of the detection procedure utilizing hybridization and kept from remaining on the surface of the carrier. If the non-complementary DNA fragments remain in the surface of the carrier, the accuracy of the detection decreases.

U.S. Pat. No. 5,387,505 describes a method of separating a target DNA fragment by binding target DNA fragments labelled with a biotin molecule with a substrate having avidin molecules.

U.S. Pat. No. 5,094,962 discloses a detection tool for a ligand-receptor assay in which receptor molecules are bonded to a porous polymer particle having a reactive group.

### SUMMARY OF THE INVENTION

It is an object of the present invention to provide a solid carrier to which a group of nucleotide derivatives or their analogues (e.g., oligonucleotides, polynucleotides, and peptide-nucleotides, which serve as probes for detecting complementary DNA fragments by way of hybridization) are attached and which is favorably employable for detecting, with high sensitivity, complementary nucleic acid fragments.

It is another object of the invention to provide a DNA chip which is employable in the procedure for detecting complementary DNA fragments without performing in advance a blocking procedure, that is, a procedure of inactivating the solid carrier in the areas having no probes, so as to keep non-complementary DNA fragments from fixing on the carrier by non-hybridization mechanism.

The present invention resides in an element comprising a solid carrier and a group of nucleotide derivatives or their analogues which are fixed to the solid carrier via covalent bonding, in which the covalent bonding contains a boron-containing ring structure.

The boron-containing ring structure formed on the element of the invention can be favorably produced by reaction of a boron-containing group and a divalent functional group, the boron-containing group being attached to the nucleotide derivatives or their analogues or to the solid carrier, while the divalent functional group being attached to the solid carrier or to the nucleotide derivatives or their analogues, respectively.

The detection method of the invention for oligonucleotides or polynucleotides such as DNA fragments can be performed by bringing the solid carrier having probes (i.e., a group of nucleotide derivatives or their analogues) fixed onto its surface into contact with oligonucleotides or polynucleotides (such as target DNA fragments) which are complementary to the probes of nucleotide derivatives or their analogues fixed onto the surface of the solid carrier in the presence of an aqueous solvent, so as to combine the complementary oligonucleotides or polynucleotides with the nucleotide derivatives or their analogues. It is preferred that the probe compound has the boron-containing group and the solid carrier has the reactive divalent group on its surface.

### DETAILED DESCRIPTION OF THE INVENTION

[Solid Carrier]

The solid carrier can be any of known solid carriers or their equivalent materials, for instance, a glass plate, a resin plate, a metal plate, a glass plate covered with polymer coat, a glass plate covered with metal coat, and a resin plate covered with metal coat. Also employable is a SPR (surface plasmon resonance) sensor plate which is described in Japanese Patent Provisional Publication No. 11-332595. CCD is also employable as described in Nucleic Acids Research, 1994, Vol.22, No.11, 2124-2125.

The solid carrier should have on its surface a plurality of boron-containing groups (such as in the form of a chain which is fixed at one end onto the surface of the solid carrier and has a boron-containing group at another end or its vicinity) or a plurality of divalent groups which are reactive to a boron-containing group to form a ring structure containing a boron atom as a ring member.

The boron-containing group is preferably derived from a boronic acid, a boronic acid ester, a boric acid, or a boric acid ester.

The boric acid ester group can be derived from trimethoxyborane, triethoxyborane, or trioctylborane. The boronic acid group can be derived from an alkylboronic acid, an alkenyl boronic acid, or an arylboronic acid. The boronic acid ester group can be derived from an alkylboronic acid ester, an alkenyl boronic acid ester, or an arylboronic acid ester.

In more detail, the boronic acid group can be preferably derived from 3-aminophenylboronic acid, 2-formylphenylboronic acid, 2-carboxyphenylboronic acid, 4-(2-carboxyethyl)phenylboronic acid, 4-carboxyphenylboronic acid, 2-(carboxyvinyl)phenylboronic acid, 3-(carboxyvinyl)phenylboronic acid, 4-(carboxyvinyl)phenylboronic acid, 3-formylphenylboronic acid, 3-formylfurane-2-boronic acid, 4-formylphenylboronic acid, 3-formylthiophene-2-boronic acid, 4-hdyroxyphenylboronic acid, and 4-vinylphenylboronic acid.

The boronic acid ester group can be preferably derived from a 3-aminophenylboronic acid ester, a 2-formylphenylboronic acid ester, a 2-carboxyphenylboronic acid ester, a 4-(2-carboxyethyl)phenylboronic acid ester, a 4-carboxyphenylboronic acid ester, a 2-(carboxyvinyl)phenylboronic acid ester, a 3-(carboxyvinyl)phenylboronic acid ester, a 4-(carboxyvinyl)phenylboronic acid ester, a 3-formylphenylboronic acid ester, a 3-formylfurane-2-boronic acid ester, a 4-formylphenylboronic acid ester, a 3-formylthiophene-2-boronic acid ester, a 4-hdyroxyphenylboronic acid ester, and a 4-vinylphenylboronic acid ester.

The boric acids and boric acid esters can be commercially obtained.

The boronic acids and boronic acid esters can be prepared in the known manners described, for instance, in Taku Ito, Kichiro Uchimoto, Akira Nakamura, Manobu Hidai, “Cheimical Reviews-17, Organic Chemistry of Former Periodical Transition Metal” edited by Hiroshi Yamazaki, Gakkai Publishing Center, 1993; Jiro Tsuji, “Organic Synthesis Developed Using Transition Metal, Its Various Reaction Modes & New Development”, Kagaku Dojin Co., Ltd. 1997; L. Brandsma, S. F. Vasilevsky, H. D. Verkruijsse, “Application of Transition Metal Catalysts in Organic Synthesis” Springer, 1998; and “Organic Synthesis in Water” edited by Paul A. Grieco, Blackie Academic & Professional 1998.

The divalent group which is able to form a boron-containing structure upon reaction with the boron-containing group can be a diol group, a diamine group, or an aminoalcohol group, such as a group derived from 1,2-diol, 1,3-diol, 1,2-aminoalcohol, 1,3-aminoalcohol, 1,2-diamine, 1,3-diamine, 2-hydroxycarboxylic acid, 2-amino-carboxylic acid, 3-hydroxycarboxylic acid, or 3-amino-carboxylic acid.

Representative examples of the divalent groups are shown by the following structures:

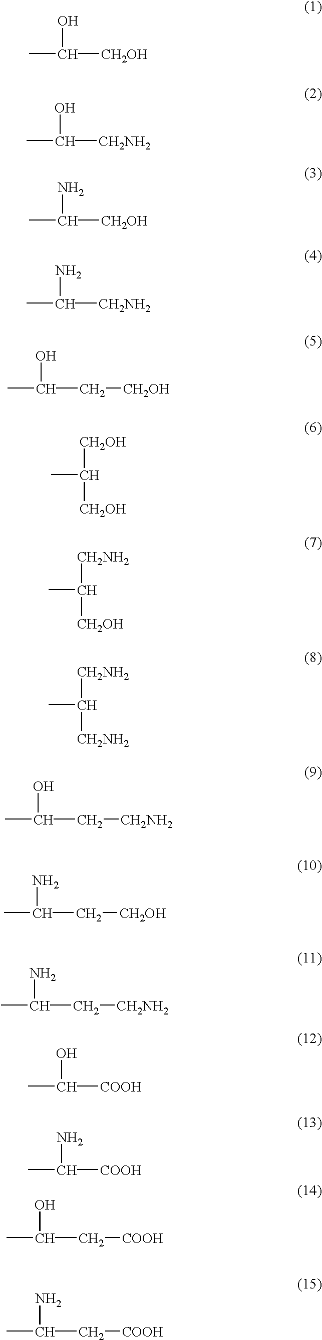

The boron-containing group can be fixed onto the solid carrier in the following manner.

In the case that a solid carrier having hydroxyl groups on its surface such as a glass plate or a plate coated with a polymer having alcoholic hydroxyl groups is employed, a boron-containing compound attached to a silane-coupling agent can be brought into contact with the hydroxyl groups on the carrier so as to perform a reaction of the hydroxyl groups with amino groups of the silane-coupling agents. For instance, 4-(2-carboxyethyl)phenylboronic acid (i.e., boronic acid ester, available from Lancaster Synthesis Corp.) is subjected to dehydration reaction with 1,3-propanediol to give a six-membered cyclic ester, which is then treated with a condensing agent such as carbodiimide in the presence of triethylamine and 3-aminopropyltrimethoxysilane, to produce 4-(2-(3-trimethoxysilylpropylcarbamoyl)ethyl)phenylboronic acid 1,3-propanediol ester. The resulting ester is then brought into contact with the solid carrier, to give a solid carrier on which a boronic ester is fixed.

In the case that a solid carrier having amino groups on its surface (for instance, a glass plate having been treated with 3-aminopropyltrimethoxysilane, or a glass plate coated with a amino group-containing polymer) is employed, a carboxyl-containing boric acid ester, a carboxyl-containing boronic acid, or a carboxyl-containing boronic acid ester is fixed onto the solid carrier by producing an amide bonding using a condensing agent (e.g., a carbodiimide compound). For instance, 4-(2-carboxyethyl)phenylboronic acid (available from Lancaster Synthesis Corp.) and a condensing agent such as carbodiimide are simultaneously brought into contact with the carrier to fix a boronic acid onto the carrier.

Otherwise, a boron-containing compound (e.g., boric acid ester, boronic acid, or boronic acid ester) having an aryloxycarbonylamino group is brought into contact with the solid carrier, which is then heated in the presence or absence of a base, to form a urea bonding.

A group reactive to an amino group can be formyl, sulfo, isocyanato, isothiocyanato, or acid anhydride. In the procedure for fixing a boron-containing compound, heating, treating with a base, and/or treatment with a condensing agent can be utilized.

A glass plate having on its surface a divalent functional group (divalent partial structure) which is reactive with the boron-containing group to form a ring structure can be prepared by coating an aqueous solution of polyvinyl alcohol or a polymer having as its recurring unit N-[tris(hydroxymethyl)methyl]acrylamide on the surface to form a thin layer of the polymer. Alternatively, the glass plate is treated with tetramethoxysilane to form on its surface a silyl ether group in advance of the coating with an aqueous polymer solution. Otherwise, the glass plate having on its surface a silyl ether group formed by treatment with tetramethoxysilane may be dipped in an aqueous polymer solution.

[Probe Compound—Nucleotide Derivative or its Analogue]

A probe compound (which is a nucleotide derivative or its analogue, such as polynucleotide, oligonucleotide, PNA, or one of their analogues) having at its one terminal a divalent functional group (divalent partial structure) which is reactive with the boron-containing group to form a ring structure can be prepared by one of the following two methods.

(1) A primer which is a probe compound having an appropriate divalent functional group is multiplied by the PCR method.

(2) A primer which is a probe compound having a reactive group such as amino is multiplied by the PCR method, and to the resulting probe compounds having a reactive group is attached an appropriate divalent functional group.

Generally, the latter method can be readily performed, and accordingly is preferred in the present invention. The attachment of an amino group to the probe compound can be attained by forming an amide bonding between the amino group and a carboxyl group of an appropriate compound using an appropriate condensing agent.

In more detail, the probe compound having an amino group at its terminal can be reacted with a compound having as its partial structure a reactive compound such as 1,2-diol, 1,3-diol, 1,2-aminoalcohol, 1,3-aminoalcohol, 1,2-diamine, 2-hydroxycarboxylic acid, 2-aminocarboxylic acid, 3-hydroxycarboxylic acid or 3-amiocarboxylic acid, to combine the structure of reactive compound with the probe compound. A probe compound having a thiol group at is terminal also can be favorably employed.

A preferred compound having the divalent functional group which is favorably employed for forming the amido bonding with the amino group of a probe compound can be N-(2-hydroxyethyl)glycine, N,N-bis(2-hydroxyethyl)glycine, trimethylol-acetic acid, 2,2-dimethylol-propionic acid, gluconic acid, glyceridic acid, serine, homoserine, N-(2-hydroxyethyl)aspartic acid, 2-ketoglutaconic acid, pantothenic acid, threonic acid, tricin, or (N-[tris-(hydroxymethyl)methyl]glycine. The formation of amido bonding can be attained using a condensing agent.

The amino group of the probe compound can be alkylated so as to give a divalent functional partial structure which is able to produce the bonding in the form of a ring. A compound for giving the divalent functional partial structure can be ethylene oxide, 2-chloroethanol, 2-bromoethanol, 3-chloropropanol, 3-bromopropanol, 3-bromo-1,2-propanediol, glycidol, 3-bromo-2,2-dimethylpropanol, or epoxysuccinic acid. Glycidol and epoxysuccinic acid are most preferred.

The thiol group of the probe compound can be alkylated so as to give a divalent functional partial structure which is able to produce the bonding in the form of a ring. A compound for giving the divalent functional partial structure can be 3-chloro-1,2-propanediol, 3-bromo-1,2-propanediol, glycidol, or epoxysuccinic acid.

The reaction for alkylating the terminal functional group can be generally performed by heating a mixture of a probe compound having its terminal the functional group and an alkylating reagent, preferably in the presence of a base. The presence of a base accelerates rate of the reaction. The base can be one of those described below. A probe compound having at its terminal a boron-containing group can be also produced by one of the following two methods.

(1) A Method Using a Probe Compound Having at its Terminal an Amino Group.

The probe compound having at its terminal an amino group can be produced by a known method and is also available commercially. Therefore, a procedure similar to the procedure for forming a boron-containing group on the solid carrier can be employed. In more detail, the amino group of the probe compound can be combined with a coupling component having carboxyl, formyl, halosulfonyl, isocyanato or isothiocyanato or a coupling component having as its partial structure an acid anhydride or a ketene using heat treatment or using an appropriate base and a condensing agent. In this procedure, a coupling component having carboxyl, isocyanato, or isothiocyanato is preferred. Most preferred is a coupling component having carboxyl, which can form an amido bonding with amino using an appropriate condensing agent (e.g., carbodiimide compound).

(2) A PCR method Using a Primer Which is a Probe Compound Having its Terminal a Boron-Containing Group.

This process can be performed in a manner similar to the PCR method for producing probe compounds having an amino group at their terminals.

The reactions employed in the various methods described in the specification can be conducted in the presence of an acid or a base. The acid can be an inorganic acid or an organic acid, such as hydrochloric acid, sulfuric acid, phosphoric acid, p-toluenesulfonic acid, trifluoroacetic acid, acetic acid, or trifluoromethanesulfonic acid.

The base can be an organic base or an inorganic base which may be employed singly or in combination. Examples of the inorganic bases include potassium carbonate, sodium carbonate, cesium carbonate, lithium hydroxide, sodium hydroxide, potassium hydroxide, cesium hydroxide, calcium hydroxide, and magnesium hydroxide. Examples of the organic bases include trimethylamine, triethylamine, tetramethylammonium hydroxide, dimethylbenzylamine, diethylaniline, pyridine, 4-dimethylaminopyridine, 1,4-diazabicyclo[2.2.2]octane, 1,8-diazabicyclo[5.4.0]-7-undecene, sodium methoxide, sodium ethoxide, potassium t-butoxide, n-butyllithium, lithium diisopropylamide, tetrabutylammonium hydroxide, sodium acetate, and potassium acetate. The base can be employed in combination with an acid.

The reactions can be performed in an aqueous solvent or an organic solvent. The organic solvent may be a hydrophobic solvent such as toluene, xylene or n-hexane. However, a polar solvent which is miscible with water can be preferably employed. Examples of the preferred polar solvents include ethyl acetate, methyl acetate, methanol, ethanol, isopropyl alcohol, n-butanol, t-butanol, sulforane, 1,2-diemethoxyethane, dimethylformamide, dimethylsulfoxide, dimethylacetamide, acetonitrile, propionitrile, diethyl ether, tetrahydrofuan, ethylene glycol, 1,3-propanediol, 1,4-butanediol, glycerol, 2-methoxyethanol, diethylene glycol, diethylene glycol dimethyl ether, acetic acid, pyridine, formic acid, propionic acid, and valeric acid.

The probe compounds, namely, nucleotide derivatives or their analogues to be fixed to the solid carrier can be oligonucleotides, polynucleotides, or peptide-nucleotides. A DNA fragment can be employed as the probe compound.

The nucleotide derivative may be polynucleotide such as cDNA, a portion of cDNA, or EST. The polynucleotide is favorably employed for studying gene expression. Otherwise, nucleotide derivatives to be fixed onto the solid carrier may be oligonucleotides, which are favorably employed for studying variations and polymorphism of gene. The oligonucleotide to be fixed onto the solid carrier preferably is one of 3 to 50-mers, more preferably 10 to 25 mers. The oligonucleotide and polynucleotide can have one or more substituent groups and/or cross-linking groups, provided that the attachment of these groups does not impart adverse influence to the function of the oligonucleotide and polynucleotide. For instance, LNA (locked nucleic acid) which is described in J. Am. Chem. Soc., 1998, 120, 13252-13253, can be employed.

[Procedure of Fixing Probe Compounds]

The nucleotide derivatives (or their analogues) to be fixed on the solid carrier are dissolved or dispersed in an aqueous solution. Generally, the aqueous solution is once placed on a plastic plate having 96 or 384 wells, and then spotted onto a solid carrier using a spotting means.

The reaction for fixing the probe compounds having at their terminal (or in the vicinity) a boron-containing group or a divalent functional group to the solid carrier having on its surface a counter group can be performed at ambient temperatures or under cooling (such as 5 to 10° C.) or heating. The heating condition is favorably adopted. Preferably, the reaction is performed at 4 to 150° C., more preferably at 50 to 130° C., most preferably at 50 to 100° C. The reaction can be conducted in a pressure-resistant vessel such as an autoclave.

In order to keep the spotted aqueous solution from evaporating, it is preferred to add a high boiling-point compound to the aqueous solution containing nucleotide derivatives. The high boiling-point compound should be soluble in an aqueous medium, should not disturb hybridization procedure, and preferably has an appropriate viscosity. Examples of the high boiling-point compounds include glycerol, ethylene glycol, dimethylsulfoxide, and a hydrophilic polymer having a low molecular weight (typically, in the range of 10³ to 10⁶) such as polyacrylamide, polyethylene glycol, or poly(sodium acrylate). The high boiling-point compound preferably is glycerol or ethylene glycol. The high boiling-point compound is preferably incorporated into an aqueous nucleotide derivative solution in an amount of 0.1 to 2 vol. %, particularly 0.5 to 1 vol. %. Otherwise, the spotted aqueous solution is preferably kept at under the conditions of a high humidity (such as 90% RH or more) and an ordinary temperature (25 to 50° C.).

The aqueous solution is spotted onto the solid carrier under the condition that each drop of the solution generally has a volume of 100 pL to 1 μL, preferably 1 to 100 nL. The nucleotide derivatives preferably spotted onto the solid carrier are in an amount (number) of 10² to 10⁵/cm². In terms of mol., 1 to 10⁻¹⁵ moles are spotted. In terms of weight, several ng or less of nucleotide derivatives are spotted. The spotting of the aqueous solution is made onto the solid carrier to form several dots having almost the same shape and size. It is important to prepare these dots to have the same shape and size, if the hybridization is quantitatively analyzed. Several dots are formed separately from each other with a distance of 1.5 mm or less, preferably 100 to 300 μm. One dot preferably has a diameter of 50 to 300 μm.

After the aqueous solution is spotted on the solid carrier, the spotted solution is preferably incubated, namely, kept for a certain period at room temperature or under warming, so as to fix the spotted nucleotide derivatives onto the carrier. In the course of incubation, UV irradiation or surface treatment using sodium borohydride or a Shiff reagent may be applied. The UV irradiation under heating is preferably adopted. It is assumed that these treatments are effective to produce additional linkage or bonding between the solid carrier and the attached oligonucleotide derivatives. The free (namely, unfixed) nucleotide derivatives are washed out using an aqueous solution. Thus washed solid carrier is then dried to give a nucleotide derivative-fixed solid carrier (such as DNA chip) of the invention.

It is not necessary to subject thus prepared analytical element to blocking treatment. However, the analytical element may be subjected to blocking treatment, if desired.

The nucleotide derivative-fixed solid carrier of the invention is favorably employable for monitoring of gene expression, sequencing of base arrangement of DNA, analysis of mutation, analysis of polymorphism, by way of hybridization.

[Sample Nucleic Acid Fragment—Target]

A target DNA fragment or a sample DNA fragment, which is subjected to the analysis concerning the presence of a complementary DNA fragment can be obtained from various origins. In the analysis of gene, the target DNA fragment is prepared from a cell or tissue of eucaryote. In the analysis of genome, the target DNA fragment is obtained from tissues other than erythrocyte. In the analysis of mRNA, the target sample is obtained from tissues in which mRNA is expressed. If the DNA chip has an oligonucleotide fixed in its solid carrier, the target DNA fragment preferably has a low molecular weight. The target DNA may be multiplied by PCR method.

To the target DNA fragment is attached an RI label or a non-RI label by a known method. The non-RI label is preferably utilized. Examples of the non-RI labels include fluorescence label, biotin label, and chemical luminescence label. The fluorescence label is most preferably employed. Examples of the fluorescence labels include cyanine dyes (e.g., Cy3 and Cy5 belonging to Cy Dye™ series), rhodamine 6G reagent, N-acetoxy-N²-acetyl-aminofluorene (AAF), and AAIF (iodide derivative of AAF). The target or sample DNA fragments labelled with different fluorescence indicators can be simultaneously analyzed, if the fluorescence indicators have fluorescence spectrum of different peaks. Also employable is an electroconductive label.

[Hybridization]

The hybridization is performed by spotting an aqueous sample solution containing a target DNA fragment onto a DNA chip. The spotting is generally done in an amount of 1 to 100 nL. The hybridization is carried out by keeping the DNA chip having the spotted sample solution thereon at a temperature between room temperature and 70° C., for 6 to 20 hours. After the hybridization is complete, the DNA chip is washed with an aqueous buffer solution containing a surface active agent, to remove a free (namely, unfixed) sample DNA fragment. The surface active agent preferably is sodium dodecyl sulfate (SDS). The buffer solution may be a citrate buffer solution, a phosphate buffer solution, a borate buffer solution, Tris buffer solution, or Goods buffer solution. The citrate buffer solution is preferably employed.

The present invention is further described by the following examples.

### EXAMPLE 1

### Manufacture of Oligonucleotide-Fixed Plates

(1) Preparation of Glass Plate Having on its Surface Divalent Functional Groups Which React with Boron-Containing Groups to Form Boron-Containing Rings

A slide glass (25 mm×75 mm) was immersed in an ethanol solution of 2 wt. % 1,2-bis(triethoxysilyl)ethane (silane coupling agent, available from Aldrich Corp.) for 10 minutes. Subsequently, the slide glass was taken out, washed with ethanol, and dried at 110° C. for 10 min. Thus, a silane coupling agent-treated slide glass (A) was prepared.

The silane coupling agent-treated slide glass (A) was then immersed in 50 mL of a 4 wt. % solution of polyvinyl alcohol (polymerization degree: approx. 2,000, saponification value: 80%, available from Tokyo Kasei Industries, Co., Ltd.) for one hour. Subsequently, the slide glass was taken out of the solution, washed with acetonitrile, and dried for one hour under reduced pressure, to prepare a glass plate (C) having polyvalent hydroxyl groups on its surface.

(2) Fixation of Oligonucleotide and Measurement of Fluorescence Strength

An oligonucleotide (3′-CTAGTCTGTGAAGTGTCTGATC-5′, 22-mers) having an amino group at 3′-terminal and a fluorescent label (FluoroLink, Cy 5-dCTP, available from Amasham Pharmacia Biotec Corp.) at 5′-terminal was treated with 4-(2-carboxyethyl)phenylboronic acid (available from Lancaster Synthesis Corp.) and 1-ethyl-3-(3-dimethylaminopropyl)carbodiimide hydrochloride (available from Tokyo Kasei Industries Co., Ltd.) to incorporate a boronic acid group into the 3′-terminal of the oligonucleotide.

The oligonucleotide having a boronic acid group at its 3′-terminal was dispersed in 1 μL of an aqueous solution containing a carbonate buffer solution (0.1 M, pH 9.8) at a concentration of 1×10⁻⁶ M. The buffer solution was then spotted onto the glass plate (C) obtained in (1) above, and this was immediately kept at 60° C., 90% RH for one hour. Thus treated glass plate was then washed successively twice with a mixture of aqueous 0.1 wt. % SDS (sodium dodecyl sulfate) solution and aqueous 2×SSC solution (obtained by twice diluting standard sodium chloride-citrate buffer solution (SSC)), and once with the aqueous 0.2×SSC solution. Thus washed glass plate was placed in an aqueous 0.1 M glycine solution (pH 10) for 1.5 hours, washed with distilled water, and then dried at room temperature, to obtain a glass plate (D1) on which the oligonucleotides were fixed.

The fluorescence strength of thus treated plate (D1) was measured using a fluorescence scanning apparatus. The fluorescence strength was 1,670, which was well higher than the background fluorescence strength. This means that the oligonucleotides are well fixed onto the glass plate.

### EXAMPLE 2

### Detection of Target Oligonucleotide

(1) Preparation of DNA Chip

A DNA chip, namely, glass plate (D2) on which the oligonucleotides were fixed was prepared in the same manner as in Example 1-(1) except for using the oligonucleotide having no fluorescent label.

(2) Detection of Target Oligonucleotide

A target oligonucleotide (GATCAGACACTTCACAGACTAG-5′, 22-mers) having Cy5 (fluorescent label) at its 5′-terminal was dispersed in 20 μL of a hybridizing solution (mixture of 4×SSC and 10 wt. % SDS). The resulting solution was spotted onto the glass plate (D2) prepared in (1) above, and its spotted surface was covered with a covering glass. Thus covered chip was subjected to incubation at 60° C. for 20 hours in a moisture chamber. The incubated chip was washed successively with a mixture of 0.1 wt. % SDS and 2×SSC, a mixture of 0.1 wt. % SDS and 0.2×SSC, and an aqueous 0.2×SSC solution, centrifuged at 600 r.p.m. for 20 seconds, and dried at room temperature.

The fluorescence strength of thus treated glass plate was measured using a fluorescence scanning apparatus. The fluorescence strength was 690, which was well higher than the background fluorescence strength. This means that the target oligonucleotides are well fixed to the DNA chip having the complementary oligonucleotide probe.

What is claimed is:

1. An element comprising a solid carrier and a group of nucleotide derivatives or their analogues which are fixed to the solid carrier via covalent bonding, in which the covalent bonding contains a boron-containing ring structure.

2. The element of claim 1, wherein the boron-containing ring structure is produced by reaction of a boron-containing group and a divalent functional group, the boron-containing group being attached to the nucleotide derivatives or their analogues or to the solid carrier, while the divalent functional group being attached to the solid carrier or to the nucleotide derivatives or their analogues, respectively.

3. The element of claim 2, wherein the boron-containing group is derived from a boronic acid, a boronic acid ester, a boric acid, or a boric acid ester.

4. The element of claim 2, wherein the boron-containing group is a boric acid ester group which is derived from trimethoxyborane, triethoxyborane, or trioctylborane.

5. The element of claim 2, wherein the boron-containing group is a boronic acid group which is derived from an alkylboronic acid, an alkenyl boronic acid, or an arylboronic acid.

6. The element of claim 2, wherein the boron-containing group is a boronic acid ester group which is derived from an alkylboronic acid ester, an alkenyl boronic acid ester, or an arylboronic acid ester.

7. The element of claim 2, wherein the boron-containing group is a boronic acid group which is derived from a boronic acid selected from the group consisting of 3-aminophenylboronic acid, 2-formylphenylboronic acid, 2-carboxyphenylboronic acid, 4-(2-carboxyethyl)phenylboronic acid, 4-carboxyphenylboronic acid, 2-(carboxyvinyl)phenylboronic acid, 3-(carboxyvinyl)phenylboronic acid, 4-(carboxyvinyl)phenylboronic acid, 3-formylphenylboronic acid, 3-formylfurane-2-boronic acid, 4-formylphenylboronic acid, 3-formylthiophene-2-boronic acid, 4-hdyroxyphenylboronic acid, and 4-vinylphenylboronic acid.

8. The element of claim 2, wherein the boron-containing group is a boronic acid ester group which is derived from a boronic acid ester selected from the group consisting of a 3-aminophenylboronic acid ester, a 2-formylphenylboronic acid ester, a 2-carboxyphenylboronic acid ester, a 4-(2-carboxyethyl)phenylboronic acid ester, a 4-carboxyphenylboronic acid ester, a 2-(carboxyvinyl)phenylboronic acid ester, a 3-(carboxyvinyl)phenylboronic acid ester, a 4-(carboxyvinyl)phenylboronic acid ester, a 3-formylphenylboronic acid ester, a 3-formylfurane-2-boronic acid ester, a 4-formylphenylboronic acid ester, a 3-formylthiophene-2-boronic acid ester, a 4-hdyroxyphenylboronic acid ester, and a 4-vinylphenylboronic acid ester.

9. The element of claim 2, wherein the divalent group is a diol group, a diamine group, or an aminoalcohol group.

10. The element of claim 2, wherein the divalent group is a group derived from 1,2-diol, 1,3-diol, 1,2-aminoalcohol, 1,3-aminoalcohol, 1,2-diamine, 1,3-diamine, 2-hydroxycarboxylic acid, 2-aminocarboxylic acid, 3-hydroxycarboxylic acid, or 3-aminocarboxylic acid.

11. The element of claim 2, wherein the divalent group is represented by one of the following structures:

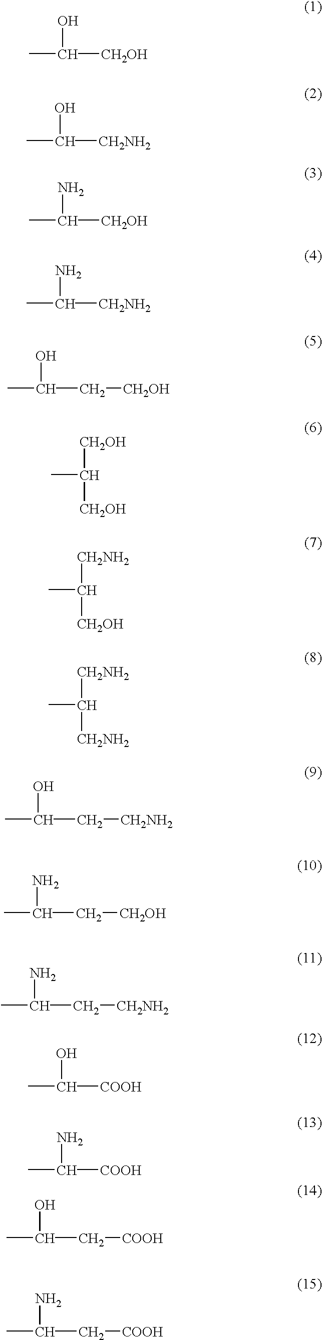

12. A method for preparing an element comprising a solid carrier and a group of nucleotide derivatives or their analogues fixed to the solid carrier via covalent bonding containing a boron-containing ring structure, which comprises bringing in a liquid phase a group of nucleotide derivatives or their analogues having at one end thereof or its vicinity a boron-containing group or a divalent group reactive to the boron-containing group to produce a boron-containing ring structure into contact with a solid carrier having thereon a divalent group reactive to the boron-containing group or a boron-containing group, respectively.
